(19) United States
(12) Patent Application Publication
(10) Pub. No.: US 2025/0228435 A1
(43) Pub. Date: Jul. 17, 2025

(54) FLEXIBLE PIPE OF INSERTION INSTRUMENT, INSERTION INSTRUMENT, AND MANUFACTURING METHOD OF FLEXIBLE PIPE OF INSERTION INSTRUMENT

(71) Applicant: OLYMPUS MEDICAL SYSTEMS CORP., Tokyo (JP)

(72) Inventors: Masato INOUE, Tokyo (JP); Hisanobu SUZUKI, Tokyo (JP)

(73) Assignee: OLYMPUS MEDICAL SYSTEMS CORP., Tokyo (JP)

(21) Appl. No.: 19/019,744

(22) Filed: Jan. 14, 2025

Related U.S. Application Data
(60) Provisional application No. 63/621,631, filed on Jan. 17, 2024.

(51) Int. Cl. A61B 1/00; A61M 25/00

(52) U.S. Cl. CPC A61B 1/00112; A61B 1/00103; A61B 1/0011; A61M 25/0012; A61M 25/005

(57) ABSTRACT

A flexible pipe for use with an insertion instrument includes: a cylinder including a first end portion and a second end portion, a first pipe sleeve attached to the first end portion; a second pipe sleeve attached to the second end portion; and a wire inserted through the cylinder. A third end portion of the wire is attached to the first pipe sleeve, and a fourth end portion of the wire is attached to the second pipe sleeve. The wire has a first elongation rate and the cylinder has a second elongation rate, the first elongation rate smaller than the second elongation rate.

### CROSS REFERENCE TO RELATED APPLICATION

This application is based on and claims priority under 35 U.S.C. § 119 to U.S. Provisional Application No. 63/621,631, filed on Jan. 17, 2024, the entire contents of which are incorporated herein by reference.

An embodiment of the present disclosure relates to a flexible pipe of an insertion instrument, an insertion instrument, and a manufacturing method of a flexible pipe of an insertion instrument.

### FIELD OF DISCLOSURE

A universal cord of an endoscope, which is a flexible pipe of an insertion instrument, is a connecting cord that connects an operation portion of the endoscope, and a connector connected to external devices (for example, a video processor and a light source apparatus).

### BACKGROUND

Japanese Patent Application Laid-Open Publication No. H11-332818 discloses a universal cord configured by a stacked structure in which a flex, a braid, and an outer skin (tube) are sequentially stacked. The flex is a spiral pipe formed by winding a strip-shaped thin plate in a spiral shape, the braid is a mesh pipe formed by braiding a metal thin wire, a synthetic fiber, or the like, and the outer skin is formed of a resin.

### SUMMARY

A flexible pipe for use with an insertion instrument includes: an elongated cylinder including a first end portion and a second end portion, a first pipe sleeve attached to the first end portion; a second pipe sleeve attached to the second end portion; and a wire inserted through the elongated cylinder. A third end portion of the wire is attached to the first pipe sleeve, and a fourth end portion of the wire is attached to the second pipe sleeve. The wire has a first elongation rate and the elongated cylinder has a second elongation rate, the first elongation rate smaller than the second elongation rate.

A manufacturing method of a flexible pipe of an insertion instrument according to an embodiment of the present disclosure, includes: fabricating a spiral pipe by winding a strip-shaped metal plate in a spiral shape in a longitudinal axis direction; fabricating a cylinder by providing a flexible tube on an outside of the spiral pipe; attaching a first pipe sleeve to a first end portion of the cylinder and a second pipe sleeve to a second end portion of the cylinder; and inserting a wire through an inside of the cylinder; and attaching a first end of the wire to the first pipe sleeve and the second end of the wire to the second pipe sleeve. The wire has a first elongation rate and the cylinder has a second elongation rate, the first elongation rate smaller than the second elongation rate.

### BRIEF DESCRIPTION OF THE DRAWINGS

FIG. 1 is a perspective view of an endoscope in an embodiment.

FIG. 2 is a perspective view of a universal cord in the embodiment.

FIG. 3 is a sectional view taken along the line III-III in FIG. 2.

FIG. 4 is an exploded perspective view of the universal cord in the embodiment.

FIG. 5 is a partial sectional view of a universal cord in a modified example of the embodiment.

FIG. 6 is a flowchart of a manufacturing method of the universal cord in the embodiment.

FIG. 7 is a view of cores and molds that are used for manufacturing a tube in a reference example.

FIG. 8A is a sectional view describing a manufacturing method of the tube in the reference example.

FIG. 8B is a sectional view describing the manufacturing method of the tube in the reference example.

FIG. 8C is a sectional view describing the manufacturing method of the tube in the reference example.

FIG. 8D is a sectional view describing the manufacturing method of the tube in the reference example.

### DETAILED DESCRIPTION

### Embodiment

Hereinafter, a universal cord of an endoscope, which is a flexible pipe of an insertion instrument, will be described with reference to drawings. The drawings based on each embodiment are schematic. The relationship between thicknesses and widths of respective parts, a ratio of a thickness of a certain part to that of another part, and the like of the respective parts in the drawings are different from the actual ones. The respective drawings include parts in which the relationships and ratios among the dimensions are different. In the drawings, illustration of some components and addition of reference signs to some components are omitted.

### <Configuration of Endoscope>

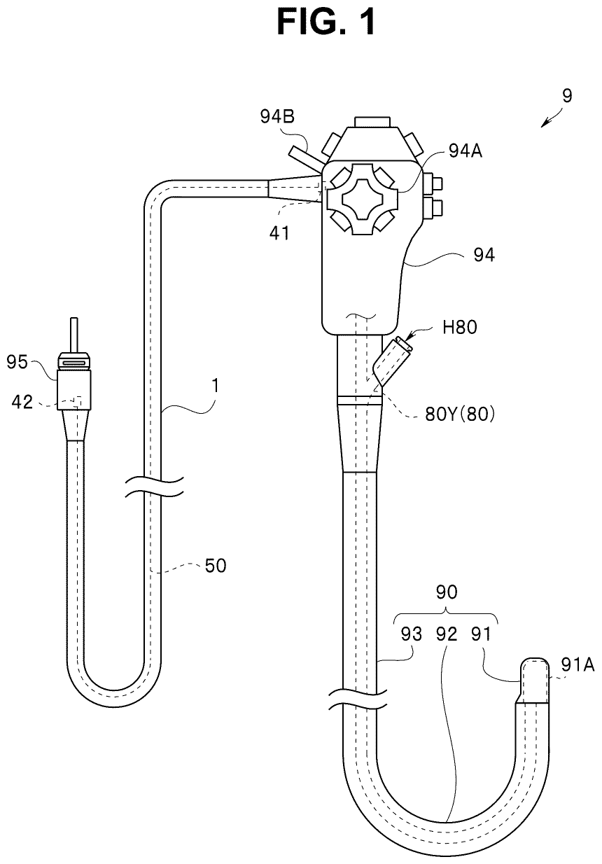

An endoscope 9 (insertion instrument) in the embodiment shown in FIG. 1 includes an insertion portion 90 configured to be inserted into a body of a subject, an operation portion 94 provided on a proximal end side of the insertion portion 90, and a universal cord 1 (flexible pipe) extended from the operation portion 94. A plurality of tubes such as a channel (suction conduit) 80, etc., and a plurality of signal lines are inserted through an inside of the universal cord 1. The flexible pipe 1 can be part of a universal cord of the endoscope 9.

The endoscope 9 can be, as one example, a single-use endoscope configured to be disposed of after having been used one time. The endoscope 9 may be used for medical purposes or industrial purposes. The single-use endoscope can be disposed of after being used once. Even single-use endoscopes may be collected after use to be cleaned, inspected, and repaired by the manufacturer to be reused in the interest of economically using resources. A single-use endoscope can be disposed of (discarded) after being used once, and may not be used multiple times. The single-use endoscope can include a single-use component and a multi-use component. The single-use component can be disposed or returned to manufacturer of after being used once, and the multi-use component can be a reusable component that can be repeatedly used. Even the reusable endoscope capable of being used multiple times by being reprocessed can be stopped being used and be sent to the manufacturer or the like for maintenance after being used a predetermined number of times.

The insertion portion 90 includes a distal end portion 91 located on the distal end side of the insertion portion, a bending portion 92 disposed on the proximal end side of the distal end portion 91, and an elongated flexible tube 93 connecting the proximal end side of the bending portion 92 and the operation portion 94, which are provided in a linked manner. Although not shown, the distal end portion 91 includes, in a rigid member 91A, an image pickup unit, an illumination optical system, and the like. The rigid member 91A is formed of a metal (for example, stainless steel) or resin (for example, epoxy resin, ABS resin, or the like).

Note that connection portions of the respective members (the distal end portion 91 and the bending portion 92, the bending portion 92 and the flexible tube 93, and the flexible tube 93 and the operation portion 94) of the insertion portion 90 are fixed by winding threads and further reinforced by a resin. The connection portions may be fixed by using pins instead of winding the threads, or may be fixed only with the resin. The connection portions of the respective members (the distal end portion 91 and the bending portion 92, the bending portion 92 and the flexible tube 93, and the flexible tube 93 and the operation portion 94) of the insertion portion 90 are fixed with pins, vis screws, rivets, etc., and are further reinforced with the resin such as an adhesive or the like. The connection portions may be fixed only with the resin, or only with pins, vis screws, rivets, etc.

The operation portion 94 includes an insertion port H80 for a treatment instrument channel that allows a treatment instrument and the like to be introduced into the insertion portion 90. As will be described later, since the channel 80 serves not only as the treatment instrument channel but also as a suction pipe, the channel 80 includes a Y-shaped branch portion 80Y.

The operation portion 94 includes various operation switches including a bending operation dial 94A. To the bending operation dial 94A, a plurality of bending operation wires (not shown) are fixed. The plurality of bending operation wires are inserted through the flexible tube 93 and respective ends of the bending operation wires are fixed to the bending portion 92. The bending portion 92 is bent in response to the rotation operation of the bending operation dial 94A.

Although not shown, a plurality of elongated protrusions are formed on an outer circumferential surface of a pipe sleeve of the flexible tube 93. The protrusions are configured to be engaged with a plurality of V-grooves formed on an inner surface of a fixing hole of the operation portion 94. Such a configuration enables a rotation direction of the bending operation dial 94A to easily coincide with a bending direction of the bending portion 92, at the time of manufacturing. Note that the V-grooves are formed such that the number thereof is greater than the number of the protrusions, to thereby enable fine adjustment between the rotation direction and the bending direction. For example, if the plurality of V-grooves are formed along the circumferential direction at a 5-degree pitch, the flexible tube 93 can be fixed to the operation portion 94 such that the rotation direction and the bending direction are adjusted with a precision within 5 degrees.

In the endoscope including, at the rigid member 91A, a raising base (forceps elevator) configured to operate a protruding direction of the treatment instrument, a raising base operation wire (not shown) for operating the raising base is inserted through the insertion portion 90 to be extended to an operation lever 94B of the operation portion 94. The rotation operation of the operation lever 94B is converted into a linear motion by a moving member located between two rails, for example. The raising base operation wire is connected to a rod that is connected to the moving member. The rod, the bending operation wire, and the raising base operation wire are configured such that the outer surfaces thereof are coated with a lubricating liquid (for example, silicone oil) in order to improve slidability with a bearing and the like.

Various wires that are inserted through the insertion portion 90 and the universal cord 1 may be inserted into a resin tube. An example of the resin tube may include, instead of an expensive fluororesin, a block copolymer composed of a rigid polyamide block and a flexible polyether block, for example. The block copolymer is relatively inexpensive and enables flexibility/elasticity to be obtained without plasticizer by controlling the composition and the ratio of the plurality of blocks.

### <Configuration of Universal Cord>

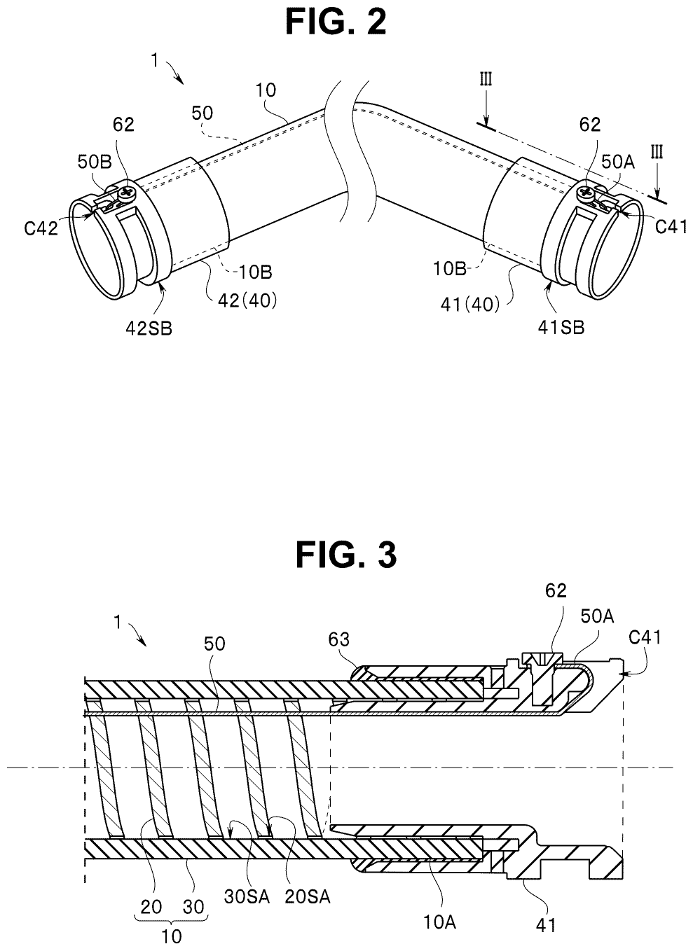

The universal cord 1 in the embodiment shown in FIGS. 2 to 4 includes a cylinder 10, two pipe sleeves 40 formed of a resin, and a wire 50. The cylinder 10 includes a spiral pipe 20 and a tube 30. The spiral pipe 20 is formed by winding a strip-shaped plate in a spiral shape, and the tube 30 is formed of a flexible resin and provided over the outer circumferential surface of the spiral pipe 20.

The cylinder 10 having an elongated cylindrical shape includes a first end 10A (first end portion) and a second end 10B (second end portion) opposite to the first end 10A. Of the two pipe sleeves 40 each having substantially a cylindrical shape, a first pipe sleeve 41 is fixed to the first end 10A of the cylinder 10 and a second pipe sleeve 42 is fixed to the second end 10B of the cylinder 10. Substantially the cylindrical shape can include a design error, manufacturing error. Substantially the cylindrical shape can include elliptical shapes and approximations of surface irregularities.

The wire 50 includes a third end 50A (third end portion) and a fourth end 50B (fourth end portion) opposite to the third end 50A. The wire 50 is configured such that the third end 50A is fixed to the first pipe sleeve 41 and the fourth end 50B is fixed to the second pipe sleeve 42. The wire 50 is inserted through an inside of the cylinder 10, and an elongation rate ΔL50 of the wire 50 is smaller than an elongation rate ΔL10 of the cylinder 10.

The flexible pipe 1 for use with the insertion instrument 9 can comprise: the cylinder 10 including the first end portion 10A and the second end portion 10B; the first pipe sleeve 41 attached to the first end portion 10A; the second pipe sleeve 42 attached to the second end portion 10B; and the wire 50 inserted through the cylinder 10. The third end portion 50A of the wire 50 can be attached to the first pipe sleeve 41. The fourth end portion 50B of the wire 50 can be attached to the second pipe sleeve 42. The wire 50 can have a first elongation rate and the cylinder 10 can have a second elongation rate, the first elongation rate can be smaller than the second elongation rate. The cylinder 10 can include: a spiral pipe 20 including a winding arranged in a spiral shape, and a tube 30 provided over the spiral pipe 20. The insertion instrument 9 can comprise the flexible pipe 1. The insertion instrument 9 can be configured to be inserted into a subject.

In the present specification, the elongation rate ΔL is calculated, at a room temperature (25° C.), based on a length L1 with no load applied and a length L2 obtained, with one end being fixed and a load of 40N being applied to the other end. In other words, the elongation rate ΔL % is calculated by ((L2−L1)/L1)×100.

The universal cord 1 is different from conventional universal cords. The universal cord 1 does not include a braid which is a mesh pipe formed by braiding a metal thin wire, a synthetic fiber, or the like. The flexible pipe 1 does not include a braided cover covering the spiral pipe 20, where the braided cover includes braided metal wire or braided synthetic fiber.

The universal cord 1 is less expensive and more excellent in flexibility than conventional universal cords with a mesh pipe. In addition, the universal cord 1 has a low torsional rigidity, thereby achieving more excellent operability than conventional universal cords. In addition, the universal cord 1 does not require the angle adjustment of the pipe sleeves located at the both ends of the universal cord, thereby reducing assembly man-hours and enabling easy manufacturing.

However, the universal cord having no mesh pipe is likely to elongate more easily due to a stress from outside and a change over time than the conventional universal cords, which will have a bad influence on the tube such as the channel and the signal lines that are inserted through the inside of the universal cord, and causes a concern of a degradation of reliability.

However, the universal cord 1 includes the wire 50 configured to restrict the elongation deformation of the cylinder 10, thereby surely achieving a flexibility, a kink-resistance, and an elongation prevention. As a result, the reliability of the universal cord is surely obtained.

Hereinafter, the detailed configuration of the universal cord 1 will be described.

The spiral pipe 20 may be formed not by a metal plate but by a rigid resin plate having elasticity. The tube 30 is formed of a flexible resin such as polyurethane, for example. The wire 50 may be a cable formed by intertwining a plurality of metal wires formed of a stainless steel, for example, since the cable thus formed is excellent in flexibility. The wire 50 may be configured such that the outer circumference thereof is covered with a resin film, and may be inserted into a resin tube having an inner diameter which is substantially equal to the outer diameter of the wire 50. Substantially equal can include a design error, manufacturing error.

The elongation rate ΔL50 of the wire 50 may be less than 10% of the elongation rate ΔL10 of the cylinder 10. If the elongation rate ΔL50 is less than 10% of the elongation rate ΔL10, the elongation deformation of the cylinder 10 can be restricted, to thereby achieve high reliability of the universal cord 1.

For example, the elongation rate ΔL10 of the cylinder 10 is 0.7% and the elongation rate ΔL50 of the wire 50 is 0.03%.

As shown in FIG. 2, the first pipe sleeve 41 includes a cutout C41 which is a first through path into which the wire 50 is inserted. The second pipe sleeve 42 includes a cutout C42 which is a second through path in which the wire 50 is inserted.

The first pipe sleeve 41 can include a first opening between an inner side surface of the first pipe sleeve 41 and an outer side surface of the first pipe sleeve 41. The third end portion 50A of the wire 50 can be located at the first opening. The second pipe sleeve 42 can include a second opening between an inner side surface of the second pipe sleeve 42 and an outer side surface of the second pipe sleeve 42. The fourth end portion 50B of the wire 50 can be located at the second opening. The wire 50 can pass through the first opening, can extend through the cylinder 10, and can pass through the second opening. The third end portion 50A of the wire 50 can be attached to the outer side surface of the first pipe sleeve 41, and the fourth end portion 50B of the wire 50 can be attached to the outer side surface of the second pipe sleeve 42.

The wire 50 is configured such that the third end 50A is fixed to an outer surface 41SB of the first pipe sleeve 41, and then the wire 50 passes through the inside of the cylinder 10 via the cutout C41, and then via the cutout C42, the fourth end 50B is fixed to an outer surface 42SB of the second pipe sleeve 42.

The wire 50 is configured such that the third end 50A is fixed to the first pipe sleeve 41, then the wire 50 is extended along the outer surface 41SB toward a side opposite to the direction in which the second pipe sleeve 42 is provided, and thereafter via the cutout C41, extended through the inside of the cylinder 10 in the direction in which the second pipe sleeve 42 is provided. The wire 50 is configured such that the fourth end 50B is fixed to the second pipe sleeve 42, then the wire 50 is extended along the outer surface 42SB toward a side opposite to the direction in which the first pipe sleeve 41 is provided, and thereafter via the cutout C42, extended through the inside of the cylinder 10 in the direction in which the first pipe sleeve 41 is provided. The wire 50 inserted through the inside of the cylinder 10 may be fixed to at least one portion on the inner surface of the cylinder 10. The third end portion 50A of the wire can extend in a first direction, the fourth end portion 50B of the wire 50 can extend at a second direction. The first direction can be opposite to the second direction. The second direction can be a direction extending from an end surface of the first pipe sleeve 41 toward a location where the first pipe sleeve 41 attaches to the first end portion of the cylinder 10. The second direction can be a direction extending from an end surface of the second pipe sleeve 42 toward a location where the second pipe sleeve 42 attaches to the second end portion of the cylinder 10. The first direction can be a direction extending from an end surface of the first pipe sleeve 41 toward a location where the first pipe sleeve 41 attaches to the first end portion of the cylinder 10. The first direction can be a direction extending from an end surface of the second pipe sleeve 42 toward a location where the second pipe sleeve 42 attaches to the second end portion of the cylinder 10.

The through paths may not be the cutouts but may be through holes. However, the cutouts allow the inserting operation of the wire 50 to be performed more easily than the through holes.

The wire 50 is fixed to each of the first pipe sleeve 41 and the second pipe sleeve 42 by each of screws 62.

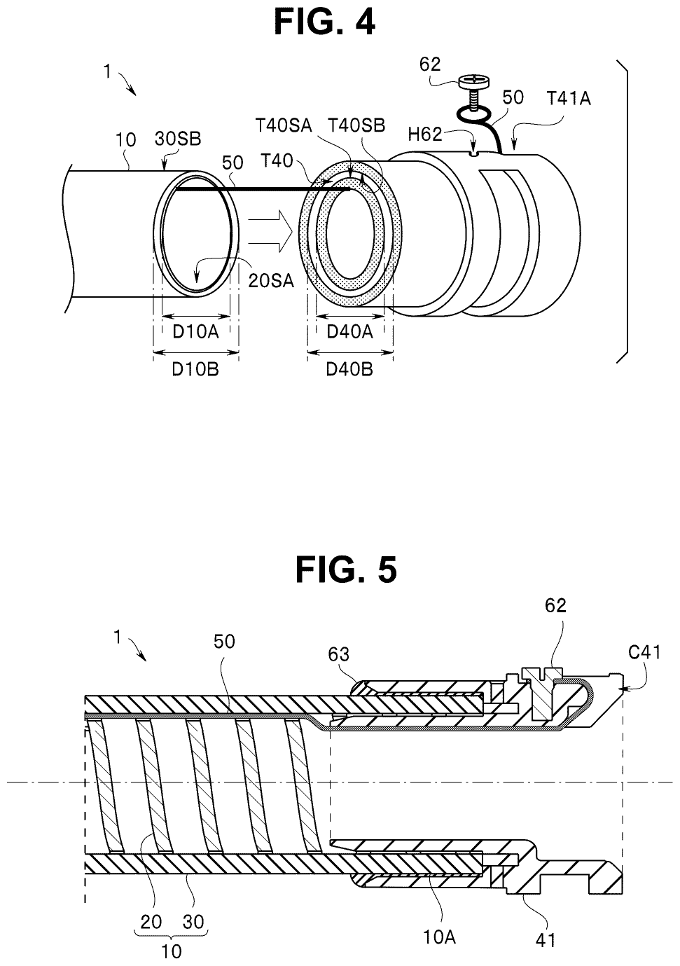

Similar to the first pipe sleeve 41 shown in FIG. 4 and FIG. 5, although not shown, the second pipe sleeve 42 also includes an annular groove T40 into which the end portion of the cylinder 10 is inserted. The groove T40 includes an inner wall surface T40SA having a diameter D40A that is slightly larger than an inner diameter D10A of the cylinder 10, and an outer wall surface T40SB having a diameter D40B that is larger than an outer diameter D10B of the cylinder 10. The first pipe sleeve 41 can include a first fixture and the second pipe sleeve 42 can include a second fixture. Each of the first fixture and the second fixture can include a screw or a pin. The third end portion 50A of the wire 50 can be attached to the first pipe sleeve 41 via the first fixture and the fourth end portion 50B of the wire 50 can be attached to the second pipe sleeve 42 via the second fixture.

When the cylinder 10 is inserted into the groove T40, the diameter of the cylinder 10 is expanded by the cylinder 10 being pushed into the groove T40, and the inner diameter D10A becomes equal to the diameter of the inner wall surface of the groove T40. Since each of the end portions of the spiral pipe 20 formed of the metal plate is inserted into the groove T40 of each of the pipe sleeves 40, the tube and the wiring inserted through the inside of the cylinder 10 are prevented from being damaged by the end surfaces of the metal plate.

Each of the two pipe sleeves 40 is formed of a transparent material, for example, a polycarbonate resin, to thereby enable the distal end positions of the cylinder 10, which are inserted respectively into the grooves T40, to be visually recognized from outside. With such a configuration, it is possible to confirm that each of the distal ends of the cylinder 10 is inserted to reach a predetermined position of the groove T40. Each of the two pipe sleeves 40 may be formed of the transparent material only in part, as long as the distal ends of the cylinder 10 inserted respectively into the pipe sleeves can be visually recognized from outside. The first pipe sleeve 41 can include a first annular groove T40 and the first end portion of the cylinder 10 can be located in the first annular groove. The second pipe sleeve 42 can include a second annular groove T40 and the second end portion of the cylinder 10 can be located in the second annular groove T40. The first end portion of the cylinder 10 located in the first annular groove T40 can be visually observable from outside the first pipe sleeve 41. The second end portion of the cylinder 10 located in the second annular groove T40 can be visually observable from outside the second pipe sleeve 42. At least a part of each of the first pipe sleeve 41 and the second pipe sleeve 42 can be formed of a transparent material.

A space between the outer diameter D10B of the cylinder 10 and the outer wall surface T40SB of each of the grooves T40 is filled with an adhesive 63. The cylinder 10 is fixed by the adhesive 63 in a state of being inserted into the groove T40 of each of the pipe sleeves 40, to thereby achieve strong fixation of the cylinder 10 to the pipe sleeves. The first end portion of the cylinder 10 can be fixed to the first annular groove T40 by adhesive. The second end portion of the cylinder 10 can be fixed to the second annular groove T40 by adhesive.

In other words, the cylinder 10, each distal end portion of which is coated with the adhesive 63, is inserted into each of the grooves T40, and thereafter the adhesive 63 is cured. The adhesive 63 is, for example, an ultraviolet curable resin that can be cured in a short time period. The respective end portions of the spiral pipe 20 and the tube 30 are fixed simultaneously to the pipe sleeves 40. The method of fixing with the adhesive is easier and less expensive than the method of fixing by brazing or with pins, etc., for example.

The pipe sleeves 40 may be bonded, by what is called caulking, to the cylinder 10. In other words, each of the end portions of the cylinder 10 is inserted into each of the grooves T40 of the pipe sleeves 40, a force is applied to the outer circumference of each of the pipe sleeves 40, to plastically deform the pipe sleeves 40, to thereby cause the pipe sleeves 40 to be firmly fixed to the cylinder 10. When the pipe sleeves 40 are fixed to the cylinder 10, the bonding by caulking and the adhesive may be used in combination.

The universal cord 1 is configured such that the first pipe sleeve 41 is fixed to the operation portion 94 of the endoscope 9 and the second pipe sleeve 42 is fixed to the connector 95 that is connected to the external device. The first pipe sleeve 41 can be attached to the operation portion 94 of the endoscope 9, and the second pipe sleeve 42 can be attached to the connector 95 that can be connected to the external device.

### <Manufacturing Method of Universal Cord>

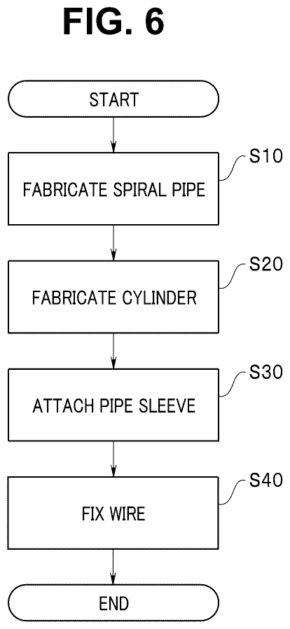

A manufacturing method of the universal cord will be described with reference to the flowchart in FIG. 6.

### <Step S10>

The strip-shaped metal plate is wound in a spiral shape in the longitudinal axis direction, to thereby fabricate the elongated spiral pipe 20.

### <Step S20>

The spiral pipe 20 is inserted through the tube 30 having flexibility, to fabricate the cylinder 10. In the specific method, for example, the tube 30 having flexibility is provided on the surface layer side of the spiral pipe 20, to fabricate the cylinder 10. For example, air is blown into the tube 30, and then the spiral pipe 20 is inserted into the tube 30, with the diameter of the tube 30 being expanded. The inner diameter of the tube 30 may be slightly larger than the outer diameter of the spiral pipe 20.

### <Step S30>

The pipe sleeves 40 are attached respectively to the both ends of the cylinder 10. In other words, the ends of the cylinder 10, to each of which the adhesive 63 is applied, are pushed respectively into the grooves T40 of the pipe sleeves 40, and thereafter curing of the adhesive 63 is performed. The adhesive 63 is, for example, an ultraviolet curable urethane acrylate resin. The curing is performed by an ultraviolet irradiation method or a combination method of ultraviolet irradiation and thermal curing.

### <Step S40>

The wire 50, the elongation rate of which is smaller than that of the cylinder 10, is inserted through the cylinder 10, and the third end 50A and the fourth end 50B of the wire 50 are fixed respectively to the pipe sleeves 40. In other words, the screws 62 around which the ends of the wire 50 are respectively wound are inserted respectively into the screw holes of the pipe sleeves 40. Note that the wire 50 may be fixed by a fixing tool including a vis screw or a pin, or by an adhesive.

The wire 50 is set to be a length for allowing the wire 50 to be substantially a linear state in the cylinder 10 in a state where the cylinder 10 is arranged in a linear state. In other words, the length of the wire 50 is substantially equal to the length of the cylinder 10. Substantially the linear state can include a design error, manufacturing error. In substantially the linear state, the cylinder 10 extend along a central axis of the tube 30, or can extend along an axis inclined with respect to the central axis in a radial direction or a circumferential direction. The axis can be inclined within 5 degrees.

The manufacturing method of the universal cord in the present embodiment enables easy manufacturing of an inexpensive universal cord. The manufacturing method of the flexible pipe 1 of the insertion instrument 9 can comprise: fabricating the spiral pipe 20 by winding the strip-shaped metal plate in a spiral shape in a longitudinal axis direction; fabricating the cylinder 10 by providing a tube 30 on an outside of the spiral pipe 20; attaching the first pipe sleeve 41 to a first end portion of the cylinder 10 and the second pipe sleeve 42 to a second end portion of the cylinder 10; inserting the wire 50 through the inside of the cylinder 10; and attaching the third end portion 50A of the wire 50 to the first pipe sleeve 41 and the fourth end portion 50B of the wire 50 to the second pipe sleeve 42. The wire 50 can have the first elongation rate and the cylinder can have the second elongation rate, the first elongation rate can be smaller than the second elongation rate.

Note that, as shown in FIG. 3, the universal cord 1 is configured such that the wire 50 is inserted through the inside of the inner surface 20SA of the spiral pipe 20. In order to prevent the wire 50 from moving in the inside of the spiral pipe 20, the wire 50 may be fixed to a guide member (not shown) provided in the spiral pipe 20. As already described above, the inner diameter of the tube 30 is slightly larger than the outer diameter of the spiral pipe 20. Therefore, as shown in FIG. 5, the wire 50 may be inserted between the outer surface 20SB of the spiral pipe 20 and the inner surface 30SA of the tube 30. The wire 50 can be located between an outer surface 20SB of the spiral pipe 20 and an inner surface 30SA of the tube 30.

### Reference Example

As already described above, the channel 80 (suction pipe) of the endoscope 9 includes the branch portion 80Y. In addition, the endoscope 9 is provided with, for example, an air/water feeding pipe, not shown, which is another channel conduit. The air/water feeding pipe also includes a part that connects a plurality of tubes into one, which is similar to the branch portion 80Y. If the part that connects the plurality of tubes into one (branch portion) is configured by a Y-shaped pipe formed of a metal, the cost increases. Even if the part is configured by a Y-shaped pipe formed of a resin, the assembly cost is high. Such a Y-shaped pipe will be a waste when the endoscope is disposed of. In view of the above, there has been a need to reduce the cost without the use of the Y-shaped pipe.

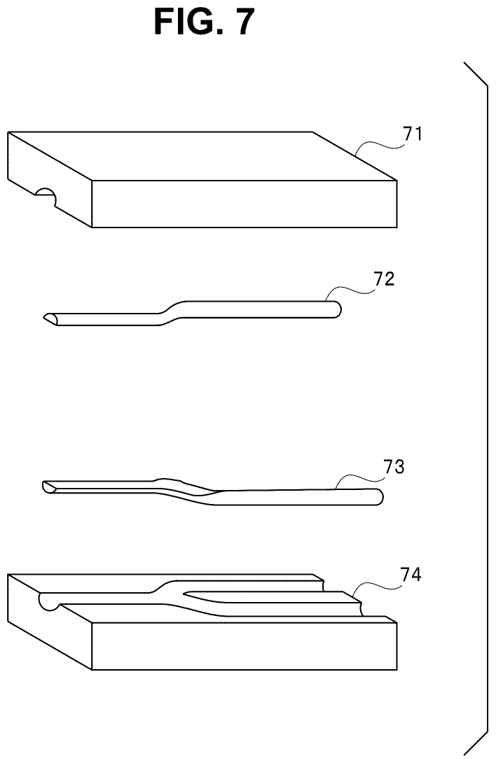

In order to manufacture the channel 81, such as the air/water feeding pipe including the branch portion, an upper mold 71, an upper core 72, a lower core 73, and a lower mold 74 are used, as shown in FIG. 7. The outer circumferential surface of a set of a pair of cores, which is formed by combining the upper core 72 and the lower core 73, has the same shape as the inner circumferential surface of the hollow portion of the channel 81. Each of the upper core 72 and the lower core 73 has a circular cross-section on one end side and a semicircular cross-section on the other end side. The upper core 72 and the lower core 73 are combined such that planes of the parts having semicircular cross-sections face each other, to be used as the set of the pair of cores.

The inner circumferential surface of a set of a pair of molds, which is formed by combining the upper mold 71 and the lower mold 74, has the same shape as the outer circumferential surface of the hollow portion of the channel 81.

### <Channel Manufacturing Method>

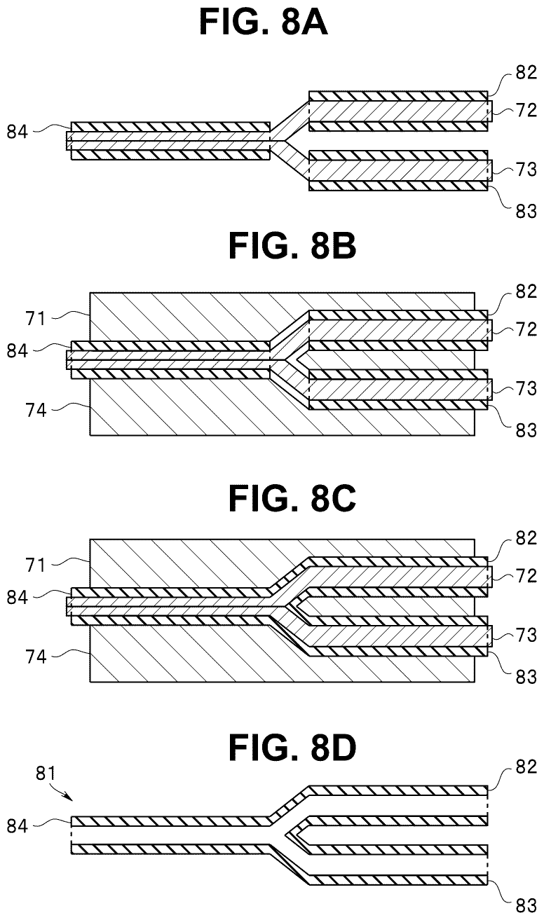

With reference to FIGS. 8A to 8D, a manufacturing method of the channel will be described. FIGS. 8A to 8D are sectional views along the longitudinal direction of the channel.

As shown in FIG. 8A, the columnar part of the upper core 72 is inserted into a first resin tube 82, and the columnar part of the lower core 73 is inserted into a second resin tube 83. The columnar part formed by combining the upper core 72 and the lower core 73 is inserted into third resin tube 84.

The first resin tube 82, the second resin tube 83, and the third resin tube 84 are formed of a resin that melts when being heated.

As shown in FIG. 8B, the set of the pair of cores over which the first resin tube 82, the second resin tube 83, and the third resin tube 84 are inserted is arranged in a space between the upper mold 71 and the lower mold 74.

As shown in FIG. 8C, when the part where the first and second resin tubes 82, 83 and the third resin tube 84 face each other is heated, the part where the tubes face each other melts and the three tubes are welded.

As shown in FIG. 8D, the upper mold 71 and the lower mold 74 are removed, and then the upper core 72 and the lower core 73 are removed, to thereby complete the channel 81 including the branch portion 80Y.

As described above, according to the manufacturing method of the channel 81, such as the air/water feeding pipe, of the endoscope 9, the first resin tube 82, which allows a first fluid to flow in the operation portion 94 from outside, and the second resin tube 83 into which the treatment instrument is inserted from the operation portion 94, are arranged in parallel. The third resin tube 84, one end of which is arranged in the distal end portion 91 of the insertion portion 90 configured to be inserted into a subject and the other end of which is arranged in the operation portion 94, is made to face the first resin tube 82 and the second resin tube 83. The upper core 72 is inserted through the first resin tube 82 and the third resin tube 84, and the lower core 73, which is to be combined with the upper core 72, is inserted through the second resin tube 83 and the third resin tube 84. The first resin tube 82, the second resin tube 83, and the third resin tube 84, through which the upper core 72 and the lower core 73 are inserted, are mounted between the upper mold 71 and the lower mold 74. Between the upper mold 71 and the lower mold 74, the part where the first and the second resin tubes 82, 83 and the third resin tube 84 face each other is heated. The part where the three tubes face each other is welded, to thereby fabricate the channel 81 including the branch portion with which the first and second resin tubes 82, 83 and the third resin tube 84 communicate with each other.

Note that the above-described manufacturing method of the channel 81 is supposed to be used for the branch portion of the air/water feeding pipe. However, a similar manufacturing method may be applied to the branch portion 80Y of the channel 80 including the suction pipe and the like.

Description has been made above by taking the universal cord of the endoscope, as an example of the flexible pipe of the insertion instrument. However, the flexible pipe may be a catheter or the insertion portion of the endoscope.

The present disclosure is not limited to the above-described embodiments, and the like, but various changes, combinations, and applications are possible without departing from the gist of the present disclosure.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.
In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.
The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.
Example 1. A flexible pipe of an insertion instrument comprising:
an elongated cylinder including a first end and a second end, the elongated cylinder including a spiral pipe formed by winding a strip-shaped plate in a spiral shape, and a tube having flexibility and provided over an outer circumferential surface of the spiral pipe;
two pipe sleeves composed of a first pipe sleeve fixed to the first end and a second pipe sleeve fixed to the second end; and
a wire including a third end and a fourth end, the third end being fixed to the first pipe sleeve, the fourth end being fixed to the second pipe sleeve, the wire being inserted through an inside of the cylinder, and having an elongation rate smaller than an elongation rate of the cylinder.
   Example 2. The flexible pipe of the insertion instrument according to Example 1, wherein the elongation rate of the wire is less than 10% of the elongation rate of the cylinder.
   Example 3. The flexible pipe of the insertion instrument according to Example 1, wherein the wire is inserted through an inside of an inner surface of the spiral pipe.
   Example 4. The flexible pipe of the insertion instrument according to Example 1, wherein the wire is inserted between an outer surface of the spiral pipe and an inner surface of the tube.
   Example 5. The flexible pipe of the insertion instrument according to Example 1, wherein the first pipe sleeve includes a first through path through which the wire is inserted, and the second pipe sleeve includes a second through path through which the wire is inserted.
   Example 6. The flexible pipe of the insertion instrument according to Example 5, wherein
the wire is configured such that the third end is fixed to an outer surface of the first pipe sleeve, the wire is inserted through the first through path, passed through the cylinder, and inserted through the second through path, and the fourth end is fixed to an outer surface of the second pipe sleeve.
   Example 7. The flexible pipe of the insertion instrument according to Example 1, wherein the wire is fixed to each of the two pipe sleeves with a fixing tool including a screw or a pin.
   Example 8. The flexible pipe of the insertion instrument according to Example 1, wherein
the wire is configured such that the third end is fixed to the first pipe sleeve, and the wire extends to a direction opposite to a direction in which the second pipe sleeve is provided, and then extends in the direction in which the second pipe sleeve is provided.
   Example 9. The flexible pipe of the insertion instrument according to Example 1, wherein
the wire is configured such that the fourth end is fixed to the second pipe sleeve, and the wire extends in a direction opposite to a direction in which the first pipe sleeve is provided, and then extends in the direction in which the first pipe sleeve is provided.
   Example 10. The flexible pipe of the insertion instrument according to Example 1, wherein the two pipe sleeves each include an annular groove into which the first end or the second end of the cylinder is inserted.
   Example 11. The flexible pipe of the insertion instrument according to Example 10, wherein the groove includes an inner wall surface having a diameter larger than an inner diameter of the cylinder and an outer wall surface having a diameter larger than an outer diameter of the cylinder.
   Example 12. The flexible pipe of the insertion instrument according to Example 11, wherein each of the two pipe sleeves is configured such that the cylinder inserted into the groove is visually recognizable from outside.
   Example 13. The flexible pipe of the insertion instrument according to Example 12, wherein at least a part of each of the two pipe sleeves is formed of a transparent material.
   Example 14. The flexible pipe of the insertion instrument according to Example 11, wherein the cylinder is fixed to the groove by an adhesive.
   Example 15. The flexible pipe of the insertion instrument according to Example 1, wherein the wire includes a cable formed by intertwining a plurality of metal wires.
   Example 16. The flexible pipe of the insertion instrument according to Example 1, wherein the insertion instrument is a single-use endoscope configured to be disposed of after being used once.
   Example 17. The flexible pipe of the insertion instrument according to Example 16, wherein
the flexible pipe is a universal cord of the endoscope, and
the flexible pipe does not include a braid which is configured to cover the spiral pipe, and is a mesh pipe formed by braiding a metal thin wire or a synthetic fiber.
   Example 18. The flexible pipe of the insertion instrument according to Example 17, wherein
the first pipe sleeve is fixed to an operation portion of the endoscope, and the second pipe sleeve is fixed to a connector that is connected to an external device.
   Example 19. An insertion instrument configured to be inserted into a subject and including a flexible pipe,
the flexible pipe comprising:
an elongated cylinder including a first end and a second end, the elongated cylinder including a spiral pipe formed by winding a strip-shaped plate in a spiral shape, and a tube having flexibility and provided over an outer circumferential surface of the spiral pipe;
two pipe sleeves composed of a first pipe sleeve fixed to the first end and a second pipe sleeve fixed to the second end; and
a wire including a third end and a fourth end, the third end being fixed to the first pipe sleeve, the fourth end being fixed to the second pipe sleeve, the wire being inserted through an inside of the cylinder, and having an elongation rate smaller than an elongation rate of the cylinder.

Example 20. A manufacturing method of a flexible pipe of an insertion instrument, comprising:
fabricating a spiral pipe by winding a strip-shaped metal plate in a spiral shape in a longitudinal axis direction;
fabricating a cylinder by providing a tube having flexibility, which extends in the longitudinal axis direction, on a surface layer side of the spiral pipe;
attaching pipe sleeves respectively to both ends of the cylinder; and
inserting a wire, which has an elongation rate smaller than an elongation rate of the cylinder, through an inside of the cylinder, and fixing both ends of the wire to the respective pipe sleeves.

What is claimed is:

1. A flexible pipe for use with an insertion instrument, comprising:
a cylinder including a first end portion and a second end portion;
a first pipe sleeve attached to the first end portion;
a second pipe sleeve attached to the second end portion; and
a wire inserted through the cylinder,
wherein a third end portion of the wire is attached to the first pipe sleeve, and a fourth end portion of the wire is attached to the second pipe sleeve, and
wherein the wire has a first elongation rate and the cylinder has a second elongation rate, the first elongation rate smaller than the second elongation rate.

2. The flexible pipe according to claim 1, wherein the first elongation rate is less than 10% of the second elongation rate.

3. The flexible pipe according to claim 1, wherein the cylinder includes:
a spiral pipe including a winding arranged in a spiral shape; and
a tube provided over the spiral pipe.

4. The flexible pipe according to claim 3, wherein the wire is located between an outer surface of the spiral pipe and an inner surface of the tube.

5. The flexible pipe according to claim 3, wherein the flexible pipe does not include a braided cover covering the spiral pipe, where the braided cover includes braided metal wire or braided synthetic fiber.

6. The flexible pipe according to claim 3, wherein the insertion instrument is an endoscope, and
wherein the flexible pipe is part of a universal cord of the endoscope.

7. The flexible pipe according to claim 6, wherein the endoscope is a single-use endoscope.

8. The flexible pipe according to claim 6, wherein the first pipe sleeve is attached to an operation portion of the endoscope, and the second pipe sleeve is attached to a connector that is connected to an external device.

9. The flexible pipe according to claim 1, wherein the first pipe sleeve includes a first opening between an inner side surface of the first pipe sleeve and an outer side surface of the first pipe sleeve,
wherein the third end portion of the wire is located at the first opening,
wherein the second pipe sleeve includes a second opening between an inner side surface of the second pipe sleeve and an outer side surface of the second pipe sleeve, and
wherein the fourth end portion of the wire is located at the second opening.

10. The flexible pipe according to claim 9, wherein the wire passes through the first opening, extends through the cylinder, and passes through the second opening,
wherein the third end portion of the wire is attached to the outer side surface of the first pipe sleeve, and
wherein the fourth end portion of the wire is attached to the outer side surface of the second pipe sleeve.

11. The flexible pipe according to claim 9, wherein the third end portion of the wire extends in a first direction,
wherein the fourth end portion of the wire extends at a second direction, and
wherein the first direction is opposite to the second direction.

12. The flexible pipe according to claim 11, wherein the first direction is a direction extending from an end surface of the first pipe sleeve toward a location where the first pipe sleeve attaches to the first end portion of the cylinder.

13. The flexible pipe according to claim 1, wherein the first pipe sleeve includes a first fixture and the second pipe sleeve includes a second fixture,
wherein each of the first fixture and the second fixture includes a screw or a pin, and
wherein the third end portion of the wire is attached to the first pipe sleeve via the first fixture and the fourth end portion of the wire is attached to the second pipe sleeve via the second fixture.

14. The flexible pipe according to claim 1, wherein the first pipe sleeve includes a first annular groove and the first end portion of the cylinder is located in the first annular groove, and
wherein the second pipe sleeve includes a second annular groove and the second end portion of the cylinder is located in the second annular groove.

15. The flexible pipe according to claim 14, wherein the first end portion of the cylinder located in the first annular groove is visually observable from outside the first pipe sleeve, and
wherein the second end portion of the cylinder located in the second annular groove is visually observable from outside the second pipe sleeve.

16. The flexible pipe according to claim 15, wherein at least a part of each of the first pipe sleeve and the second pipe sleeve are formed of a transparent material.

17. The flexible pipe according to claim 14, wherein the first end portion of the cylinder is fixed to the first annular groove by adhesive, and
wherein the second end portion of the cylinder is fixed to the second annular groove by adhesive.

18. The flexible pipe according to claim 1, wherein the wire is a cable formed by intertwining a plurality of metal wires.

19. An insertion instrument, comprising:
the flexible pipe according to claim 1,
wherein the insertion instrument is configured to be inserted into a subject.

20. A manufacturing method of a flexible pipe of an insertion instrument, comprising:
fabricating a spiral pipe by winding a strip-shaped metal plate in a spiral shape in a longitudinal axis direction;
fabricating a cylinder by providing a tube on an outside of the spiral pipe;
attaching a first pipe sleeve to a first end portion of the cylinder and a second pipe sleeve to a second end portion of the cylinder;
inserting a wire through an inside of the cylinder; and
attaching a third end portion of the wire to the first pipe sleeve and a fourth end portion of the wire to the second pipe sleeve,
wherein the wire has a first elongation rate and the cylinder has a second elongation rate, the first elongation rate smaller than the second elongation rate.
